(19) United States
(12) Patent Application Publication
(10) Pub. No.: US 2023/0037708 A1
(43) Pub. Date: Feb. 9, 2023

(54) POROUS COLLAGEN/POLYMER MATRIX BIOCOMPOSITE MATERIAL AND USE THEREOF AS AN IMPLANT FOR REPAIRING MENISCAL LESIONS OF THE KNEE AND/OR FOR PREVENTING OR TREATING OSTEOARTHRITIS OF THE KNEE

(71) Applicant: Michel ASSOR, Marseille (FR)

(72) Inventor: Michel ASSOR, Marseille (FR)

(21) Appl. No.: 17/789,324

(22) PCT Filed: Jun. 12, 2020
(86) PCT No.: PCT/FR2020/051011

§ 371 Date: Jun. 27, 2022

(30) Foreign Application Priority Data
Dec. 26, 2019 (FR) FR1915642

(51) Int. Cl. A61L 27/48; A61L 27/56; A61L 27/58; A61L 27/54

(52) U.S. Cl. CPC A61L 27/48; A61L 27/56; A61L 27/58; A61L 27/54; A61L 2430/06; A61L 2420/02

(57) ABSTRACT

A porous biocomposite material including a polymer matrix having pores defined by several surfaces and collagen on the surface of the pores and the outer surfaces of the polymer matrix, the ratio, by weight, collagen to polymer matrix is from 20:80 to 40:60. The polymer matrix of the porous biocomposite material includes a copolymer which is prepared from a poly(ε-caprolactone) diol, a poly(lactide-co-glycolide) diol and a lysine diisocyanate (LDI). Also included are an implant which is a biodegradable, porous foam and with similar biomechanics to the normal meniscus, with tensile, compressive and tear strength, and preventing the pores from collapsing under condyle-tibia pressure. It serves as a scaffold for damaged meniscus repair or replacement, indicated for grade 3 or 4 terminal knee arthrosis, for the prevention of treatment, by cartilage regeneration, of advanced knee arthrosis, to avoid knee prostheses in young patients.

### TECHNICAL FIELD

The invention relates to a porous biocomposite material comprising a polymer matrix having pores defined by several surfaces and collagen which completely or partially covers the surface of the pores and the outer surfaces of the polymer matrix.

The invention also relates to a damaged knee meniscus replacement or repair implant, comprising said porous biocomposite material.

The invention relates generally to the technical field of surgical implants designed for damaged knee meniscus repair or replacement, and more specifically, to the field of porous polymer materials suitable for use as meniscus substitutes.

### BACKGROUND

The medial and lateral menisci are fibrocartilaginous joints or tissues placed between the condyle and tibia, in the shape of a C and triangular in the shape of a wedge in a transversal cross-section. They are essential for load transmission and distribution (compression, traction). Trauma- or arthrosis-induced meniscal lesions are very common, and due to the poor vascularisation thereof, spontaneous healing is poor, and a surgical procedure involving more or less partial arthroscopic meniscectomy is most frequently necessary, despite the importance of the meniscus, with mediocre clinical outcomes. A direct correlation between residual meniscal mass and joint cartilage lesions has been demonstrated (1, 2). At the present time, there is no effective treatment in the long term for replacing significant (over 50%) meniscal loss. Meniscal allograft treatment is not reliable in the long term, with limited cellular infiltration, limited availability, problem of preservation and of disease transmission. Advances in tissue engineering have led to the production of acellular porous materials (or scaffolds) which are useful for promoting tissue colonisation and which are suitable for use in surgical clinical practice as meniscal lesion repair implants. The two best-known meniscal implants are the bovine Achilles tendon-based meniscal implant Menaflex® or CMI® (Collagen meniscal implant, Stryker) (3); and the polyurethane foam-based meniscal implant Actifit® (4).

The present Applicant has a significant meniscal implant implantation activity in grade 4 arthrosis of the knee with cell therapy and treatment of mechanical knee lesions, with several hundred fitted since 2012, in this indication of terminal arthrosis in still young subjects to avoid knee prostheses. The Actifit® implant was used, with the results presented to the SFA congress in 2017 (14). This implant was withdrawn from the market in March 2017 for financial reasons but also for toxicity problems. The CMI® implant was used from April 2017. However, these types of implants, especially CMI®, are fragile, tearing easily, with poor compressive and tensile strength, and requiring an intact meniscal wall, to withstand peripheral tensile forces. The studies report contradictory results, with variable presence of fibrocartilaginous tissue, incomplete or absence fibrocartilaginous tissue colonisation in the pores of extruded implants, and particularly for CMI®, implant collapse, partial or total resorption, continuation of the arthrosic process, and problem in respect of post-operative sepsis, cross-linked cytotoxic by-products, and allergy to animal proteins for CMI. These two products have an amorphous structure unsuitable for meniscus replacement. The Actifit® implant, withdrawn from the market, has in the polyurethane structure thereof aliphatic isocyanate families which are toxic, and which are subject to monitoring and are soon to be banned under REACH (Registration, Evaluation, Authorisation and Restriction of Chemicals): (https://echa.europa.eu/fr/substance-information/-/substanceinfo/100.023.512).

The Menaflex CMI® meniscal implant (Stryker), in particular, has unfortunately proven to be unsuitable for the indication thereof as a meniscus replacement: the fragility thereof is such that during the intra-articular fixation thereof by means of a Fastfix® Smith® and Nephew®, or Air® system from the company Stryker, the thrust of the suture needle causes the collapse and folding of the implant, which is pushed back into the tissue outside the tibial plateau (whereon it was positioned); the drilling thereof is difficult, requiring continuous rotating movements, and not direct pressure which dislocates the implant out of the tibial plateau; whereas the Actifit® polyurethane-based implant was easy to secure. Frequently, during the first sutures of the posterior part of the CMI® implant, due to the fragility, light weight, and lack of roughness thereof, this implant tends to be positioned in a vertical and non-planar manner parallel with the tibial plateau; and it frequently folds. The unsuitable texture thereof rendered the fitting and fixation thereof very difficult. Production was discontinued for the CMI® implant, which was also recently withdrawn from the market in November 2019.

Thus, at the present time, there is no effective meniscal implant on the global market.

Current tissue engineering strategies use biological materials such as collagen (5), hyaluronan (6), and silk (7), as well as synthetic polymers such as polylactic acid (8), polyglycolic acid (9), polycaprolactone (10,11) and polyethylene, polyvinyl alcohol (12). Several short-term studies have demonstrated compressive strength, fibrochondrocytic cell morphology and collagen expression, but most have failed in the longer term due to implant fracture and/or joint damage (13). And no effective meniscal implant currently exists on the market. These implants have proven to be of little use and very limited in respect of colonisation which is often incomplete or absent of fibrocartilaginous tissues in the pores of known implants, and pressure distribution between femur and tibia.

Moreover, cartilage regeneration in grade 4 knee arthrosis and the possibility of avoid knee prostheses in patients who are still young and capable, was demonstrated during a clinical trial authorised by ANSM and CPP (France) from 2010 to 2014, of one-off use of concentrated aspirated bone marrow mesenchymal stem cells (MSC), by carrying out, under arthroscopy, micro-drilling of condyle-tibia cartilage defects (faults or anomalies), by releasing the medial or lateral collateral ligament; and meeting inclusion criteria: satisfactory axis or deformation corrected by osteotomy previously or simultaneously with arthroscopy; ligamentous stability or instability treated by reconstruction previously or simultaneously; and presence of at least ⅔ of the meniscus. The results were demonstrated during scientific papers at the 2013 and 2014 Sofcot conferences (15,16).

Meniscal loss is common. It induces a risk of knee arthrosis. It represents a public health problem affecting almost 40% of the world's population. Severe grade 4 knee arthrosis is also common in young subjects with meniscal loss. Treatment by implantation under knee arthroscopy of the meniscal transplant in grade 4/4 terminal arthrosis, coupled with the treatment of arthrosis by micro-drilling of the arthrosic areas with cell therapy (PRP platelet-rich plasma concentrated platelets, or concentrated bone marrow steam cells, according to extent of the arthrosis if more than 6 cm2), and after having treated mechanical lesions in respect of misalignment by osteotomy, and in respect of ligamentous instability by ligament reconstruction; and systematic decompression release under arthroscopy of the medial or lateral collateral ligament, enables young patients of good physical fitness to avoid knee prostheses.

The scientific paper of December 2017 presented to Société Francophone d'Arthroscopie (SFA) (14) on the results of the polyurethane meniscal implant in grade 4 knee arthrosis, confirms the good results on cartilage regeneration. It also confirms the inadequate results of cell therapy and the treatment of mechanical lesions of the knee with grade 4 arthrosis if the meniscal loss of over 50% is not replaced by a meniscal implant.

Such a scientific paper of SFA confirms the key importance, for meniscal implantation under arthroscopy, of the release of the capsule and the medial or lateral collateral ligament retracted due to the loss of cartilage height. The elongation section thereof makes it possible furthermore to decompress the arthrosic compartment, reduce the complete pinching of grade 4 arthrosis by opening sufficiently to enable cartilage regeneration, which remains frequent at bone-bone contact despite any osteotomy. Above all, it greatly facilitates the fitting of the meniscal scaffold, the placement thereof and fixation thereof by multiple sutures.

The prior art can furthermore be illustrated by the patent application US20170202672A1 (Persaud, Philip A.) relating to a meniscal implant made of reinforced polycarbonate-urethane (PCU) (see ref. 17) as well as by the following studies:
study by Elsner J. et al.; 2009: relating to a Polycarbonate-Urethane meniscal implant and the use thereof in sheep knees;
study by Peng et al.; 2018: relating to a series of scaffolds made of poly (ε-caprolactone) (PCL) and of poly-lactic-co-glycolic acid (PLGA) produced by 3D printing and use thereof for alveolar bone regeneration (see ref. 19);
study by Rong Zhu et al.; 2016: relating to a series of polycarbonate urethanes (PCA) and to the evaluation particularly of the surface behaviour thereof and the biocompatibility thereof with blood (see ref. 20);
study by Vrancken, W. et al.; 2015: relating to a polycarbonate urethane meniscal implant and to the evaluation of the in vivo performances thereof in a goat model (see ref. 20);
study by Wong DY et al.; 2007: relating to the evaluation of rat brain response to degradable polymers based on (poly (L-lactic-co-glycolic acid) (PLGA) and/or poly (epsilon-caprolactone) (PCL)) (see ref. 22).

In view of the above, an aim of the invention is that of remedying the aforementioned drawbacks. In particular, one of the aims of the invention is that of proposing an alternative or enhanced implant, suitable for use for damaged medial or lateral knee meniscus repair or replacement. Moreover, a further aim of the invention is that of providing such an implant with excellent mechanical properties such as pressure resistance, flexibility, and elasticity.

### SUMMARY

The solution proposed by the invention is a porous biocomposite material comprising a polymer matrix having pores defined by several surfaces and collagen which completely or partially covers the surface of the pores and the outer surfaces of the polymer matrix. This porous biocomposite material is remarkable in that:
the polymer matrix comprises a copolymer which is the reaction product of a mixture comprising:
   a prepolymer (A) which is a poly(e-caprolactone) diol;
   a prepolymer (B) which is a poly(lactide-co-glycolide) terminated by a hydroxyl group at both ends of the molecules thereof and which has a molar ratio of lactide to glycolide ranging from 75:25 to 50:50; and
   a diisocyanate (C) which is a C1 to C4 alyl ester of lysine diisocyanate (LDI);
   and, optionally a catalyst;
the molar ratio between prepolymer (A) and prepolymer (B) in the mixture being from 10:90 to 90:10;
the molar quantity of diisocyanate (C) in the mixture being approximately once the total quantity of prepolymer (A) and prepolymer (B);
the ratio by weight of collagen to polymer matrix is from 20:80 to 40:60.

The invention also relates to an implant for the repair of a knee meniscus lesion; and/or treatment of a knee joint cartilage lesion; and/or prevention or treatment of knee arthrosis, particularly of grade 3 or 4 arthrosis of the knee; and/or regeneration of knee joint cartilage. This implant is remarkable in that it is comprises the porous composite material according to the invention. Preferentially, the implant according to the invention is embodied in a single C shape suitable for use for both the lateral meniscus and the medial meniscus.

The porous biocomposite material according to the present invention can be used readily for the manufacture of damage medial or lateral knee meniscus replacement or repair implants. It has substantially similar mechanical properties, for example rigidity and elasticity, to those of the meniscus of the knee. It also has the advantage of being biodegradable and biocompatible. Indeed, the polymer matrix of the biocompatible material of the present invention comprises polyester segments (poly-ε-caprolactone, lactide/glycolide copolymer) and di-urethane units derived from lysine linking the polyester segments to one another, the hydrolysis of this polymer matrix producing residues namely ε-hydroxyhexanoic acid, lactic acid, glycolic acid and lysine, which can all be assimilated by the body, particularly via the Krebs cycle. It will be noted that the different values of the molar ratio of prepolymer A (polycaprolactone) to prepolymer B (lactide/glycolide copolymer) and the molar ratio of lactide to glycolide in polymer matrix are suitable for adjusting the degradation rate of the biocomposite material (or implant) according to the present invention while retaining an acceptable level of mechanical properties. Moreover, the porous structure thereof impregnated with collagen offers a favourable environment for cellular growth and for fibrocartilaginous tissue formation similar to the meniscus. This porous structure, which can be produced by means of any known method in the field of polymer foam material chemistry, is furthermore well suited for serving as reservoirs for example for live cells stem cells such as mesenchymal stem cells with a view to use in knee cartilage regeneration cell therapy treatment.

The term “biocompatible” refers to materials, for example (co)polymers, which, when they are placed in contact with a biological tissue, do not negatively affect the function of this tissue (or indeed of the whole body) in any substantial way, they do not induce rejection or toxicity and do not create lesions of the biological tissues in contact therewith. In particular, the copolymers used according to the present invention, or the degradation residues thereof generated in vivo trigger no immune response, sensitivity, or irritation, or cytotoxicity, or genotoxicity. It will be noted that the di-urethane unit of such copolymers derives from a C1 to C4 alkyl ester of lysine diisocyanate (LDI). This di-urethane unit of the copolymers is non-toxic with respect to the di-urethane parts derived from conventional aliphatic isocyanates such as tetramethylene diisocyanate (TDI) and hexamethylene diisocyanate (HDI); or aromatic isocyanates such as isophorone diisocyanate.

The term “bioresorbable” denotes materials which, when they are placed in contact with biological tissue in a living body (e.g. the body of a human or animal patient), are degraded by enzymatic, hydrolytic reactions or other chemical reactions or cellular processes into by-products which are either integrated in the body, or expelled from the body. It is acknowledged that in the literature, the terms “bioresorbable”, “resorbable”, “absorbable”, “bioabsorbable” and “biodegradable” are frequently used interchangeably and such an interchangeable meaning is intended for the present application.

Further advantageous features of the invention are listed hereinafter. Each of these features can be considered alone or in combination with the remarkable features defined above, and be the subject, if applicable, of one or more divisional patent applications:
The porous biocomposite material according to the present invention is advantageously bioresorbable.
The porous biocomposite material according to the present invention is advantageously bioresorbable.
Preferentially, prepolymer (A) and prepolymer (B) are biocompatible and biodegradable and each have a molecular weight ranging from 600 g/mol to 15000 g/mol or greater than 15000 g/mol, preferably between 1000 g/mol and 10000 g/mol.
Preferentially, prepolymer (B) has a molar ratio of lactide to glycolide of 50:50.
The pore size of the polymer matrix is preferably between 25 microns and 500 microns, preferentially between 50 and 300 microns.
The polymer matrix has advantageously a porosity from 40% to 95% by volume, in particular from 74% to 85% by volume.
Preferably, the polymer matrix has a mean molecular weight greater than 100000 g/mole,
Preferentially, a mean molecular weight ranging from 250000 g/mole to 400000 g/mole.
The porous biocomposite material has advantageously a density as per the standard DIN EN ISO 845 between 0.1 g/cm³ and 0.5 g/cm³; preferentially of 0.3 g/cm³.
Preferably, the porous biocomposite material has a tear resistance of a value greater than 10 N/mm, preferentially, of a value between 20 N/mm and 25 N/mm; and a compressive strength between 2 MPa and 3 MPa; and an elongation at break of a value greater than 300%, preferentially of a value between 350% and 500%.
The collagen can be a human collagen or a non-human, for example bovine, collagen, or a combination thereof.
The collagen can be a human recombinant collagen produced in genetically modified plants, in particular a human recombinant collagen expressed by tobacco plants.
The collagen may be a type I or II collagen or a combination thereof.
The porous biocomposite material can further comprise one or more types of live cells, particularly live cells selected in the group consisting of chondroblasts, chondrocytes, stem cells, mesenchymal stem cells, adipose stem cells, where said stem cells are not human embryonic stem cells.
The porous biocomposite material can further comprise at least one bioactive agent particularly chosen in the group consisting of anaesthetic agents, opacifying agents, anti-inflammatory agents, therapeutic agents, growth factors, blood platelets.
In particular, the porous polymer matrix is presented in the form of a moulded body, preferably, a moulded body which has the shape of a lateral or medial meniscus of the knee.
Porous biocomposite material is presented advantageously in a form suitable for use as an implant for the repair of a lateral or medial meniscus lesion of the knee, and/or prevention or treatment of a joint cartilage lesion of the knee, and/or prevention or treatment of knee arthrosis, particularly of grade 3 or 4 arthrosis of the knee.

According to a further aspect, the invention also relates to the use of the biocomposite material for producing an implant for the repair of a lateral or medial meniscus lesion of the knee; and/or prevention or treatment of a knee joint cartilage lesion; and/or prevention or treatment of knee arthrosis, particularly of grade 3 or 4 arthrosis of the knee; and/or regeneration of knee joint cartilage.

According to a further aspect, the invention also relates to a method for manufacturing the biocomposite material according to the present invention. This method comprises at least the steps of:
providing a porous polymer matrix according to the invention,
applying collagen to said polymer matrix to obtain the biocomposite material.

The collagen can particularly be applied to the porous polymer matrix by soaking, chemical cross-linking, or thermal cross-linking.

Preferentially, the collagen applied to the porous polymer matrix is a type I or II human recombinant collagen or a combination thereof.

### BRIEF DESCRIPTION OF THE FIGURES

Further advantages and features of the invention will emerge more clearly on reading the following description of a preferred embodiment, with reference to the appended drawings, produced by way of indication and non-limiting examples and wherein:

FIG. 1 is a schematic top view of the porous polymer matrix or of the implant produced in the shape of a knee meniscus, according to the present invention. For reasons of clarity, the pores of the polymer matrix have not been represented;

FIG. 2 is an enlarged sectional view substantially taken along the line I-I of FIG. 1.

### DETAILED DESCRIPTION OF THE EMBODIMENTS

The present invention follows on from an in-depth study of various synthetic (co)polymers with a view to developing biocompatible and biodegradable materials having mechanical properties and a degradation rate that are optimal and being suitable for use as damaged knee meniscus replacement or repair implants.

Even though the biocomposite material according to the present invention is described hereinafter in the description with reference to the manufacture and use of a knee meniscus replacement implant, the teachings of the present description can also be applied to manufacturing and using implants for replacing other tissues of similar type and function to the meniscus, such as intervertebral disks, the temporomandibular joint (TMJ), menisci of the wrist and similar.

The present invention firstly relates to a porous biocomposite material comprising a polymer matrix impregnated and coated with collagen. The polymer matrix has a porous structure produced from a copolymer which is the reaction product of a mixture comprising (i) a prepolymer (A) which is a poly(ε-caprolactone) diol; (ii) a prepolymer (B) which is a poly(lactide-co-glycolide) terminated by a hydroxyl group at both ends of the molecules thereof; and (iii) a diisocyanate (C) which is a C1 to C4 alkyl ester of lysine diisocyanate.

Raw Material: Prepolymer A, Prepolymer B and Diisocyanate C

Prepolymer A (poly(ε-caprolactone) diol) and prepolymer B (Poly(lactide-co-glycolide) diol) used within the scope of the present invention are linear aliphatic esters terminated by hydroxyl groups. They have a molecular weight ranging from 600 g/mol to 15000 g/mol or greater than 15000 g/mol, preferably from 1000 g/mol to 10000 g/mol; more preferentially, from 1500 g/mole to 6000 g/mole. The molecular weight denotes the mean molecular weight. They have moreover a melting point within the range of about 20° C. to about 160° C., and more preferably within the range of about 30° C. to about 120° C.

Poly(ε-caprolactone) diol (prepolymer A) can be prepared by ring-opening polymerisation of the monomer ε-caprolactone, in the presence of a C2-C6 alkylene diol used as a polymerisation initiator and a suitable catalyst.

(Poly(lactide-co-glycolide) diol (prepolymer B) can be prepared from lactide and glycolide by copolymerisation, in the presence of a C2-C6 alkylene diol, used as a copolymerisation initiator, and a suitable catalyst. Lactide has two asymmetrical carbons. It can be used in racemic form (rac-lactide), or non-racemic form (LL-lactide, DL-lactide or LL/DL-lactide) or mixtures thereof.

By way of non-limiting example of C2-C6 alkylene diols suitable for use as an initiator of polymerisation (for prepolymer A) or of copolymerisation (for prepolymer B), mention can be made of 1,2-ethanediol (or ethylene glycol), 1,3-propanediol, 1,4-butanediol, 1,5-pentanediol, 1,6-hexanediol, di-ethylene glycol and tri-ethylene glycol. By way of non-limiting example of catalysts used to facilitate polymerisation or copolymerisation, mention can be made of stannous octoate and tin 2-ethylhexanoate. The quantity of catalyst (e.g. stannous octoate) used can vary within wide limits. It is generally such that the molar ratio the catalyst and the monomer ε-caprolactone for prepolymer A or the monomer lactide or glycolide for prepolymer B, is between 0.001 and 0.1, preferably between 0.002 and 0.05.

Typically, the polymerisation (for prepolymer A) and the copolymerisation (for prepolymer B) are carried out at a temperature between 25° C. and 200° C., in particular between 50° C. and 180° C. They can be carried out with or without solvent. When they are used, the solvents suitable for these polymerisation and copolymerisation reactions comprise, but without being limited thereto, chloroform, dichloromethane, THF, dioxane and DMF, toluene, xylene, and cyclohexane. Mixtures of several solvents can obviously be used. The quantity of solvent optionally used can vary within wide limits. In practice, it is such that the molar ratio between the monomer ε-caprolactone for prepolymer A or the monomer lactide or glycolide for prepolymer B and the solvent, is between 10% and 50% weight/volume, in particular between 20% and 40%. “Suitable solvent” denotes an organic solvent wherein prepolymer A and prepolymer B can be dissolved or be suspended.

The reaction time of polymerisation (for prepolymer A) and of copolymerisation (for prepolymer B) is particularly dependent on the temperature and on the presence of a solvent or not. It can vary from 30 min to one or more hours or even one or more days (for solvent-free reactions). Tracking of the progress of the reaction by sampling and evaluating the product formed by the proton nuclear magnetic resonance CH NMR) and/or Fourier Transform Infrared (FTIR) spectroscopy technique enables those skilled in the art to readily determine the most suitable reaction time for the conditions used.

Poly(ε-caprolactone) diol (prepolymer A) is moreover commercially available under the names CAPA® (Perstorp, Sweden) or Ingevity CAPA® (Tri-iso, USA).

Poly(ε-caprolactone) is degraded in vivo by hydrolysis of the ester bonds thereof and the degradation products are essentially ε hydroxyhexanoic acid which is an endogenous compound.

Prepolymer B can for example be prepared with a similar method to that described in the international application WO2000025826A1 (see example 1).

The molar lactide/glycolide ratio in prepolymer B used for preparing the polymer matrix according to the present invention can be from 75:25 to 25:75. Preferably, this molar lactide/glycolide ratio is 50:50.

Poly(lactide-co-glycolide) is degraded by hydrolysis of the ester bonds thereof and the degradation products are lactic and glycolic acids which are endogenous compounds.

Diisocyanate (C) used within the scope of the present invention is a C1 to C4 alkyl ester of lysine diisocyanate (LDI), the latter can be prepared with any known method in isocyanate chemistry, particularly with the method described in the patent document U.S. Pat. No. 3,367,920 (Merck and Co Inc). The lysine by way of raw material for preparing diisocyanate (C), can be L or S (+)-lysine; D or R(−)-lysine or a mixture thereof. The C1 to C4 alkyl group of diisocyanate (C) can be chosen in the following group: methyl, ethyl, propyl, iso-propyl, and butyl.

Preparation of the Porous Polymer Matrix

The method for manufacturing the porous polymer matrix according to the present invention comprises the following steps:
Step a): copolymerisation of prepolymer A and prepolymer B in the presence of diisocyanate C to form a copolymer (polyurethane) comprising polyester segments (poly-ε-caprolactone, lactide/glycolide copolymer) joined by di-urethane units derived from lysine diisocyanate (LDI)
Step b): generation of pores in the copolymer (polyurethane) obtained in step a) to obtain the porous structure of the polymer matrix.

In step a) a mixture comprising prepolymer A poly(ε-caprolactone) diol) and prepolymer B (Poly(lactide-co-glycolide) diol) is reacted, in a suitable solvent and at a temperature from 25° C. to 180° C., with diisocyanate C (lysine diisocyanate (LDI)), and if applicable in the presence of a suitable catalyst, the molar quantity of diisocyanate (C) in the mixture being about once (1 to 1.03 times) the total molar quantity of prepolymer (A) and prepolymer (B) and the molar ratio between prepolymer (A) and prepolymer (B) in the mixture being from 10:90 to 90:10.

“Suitable solvent” denotes an organic solvent wherein prepolymer A, prepolymer B and the copolymer to be formed can be dissolved or be suspended.

Step a) can be performed in the presence of a catalyst chosen from the group comprising dibutyltin dilaurate, stannous octoate, tin 2-ethylhexanoate, and zinc 2-ethyl hexanoate.

Step (a) is preferably conducted at a temperature between 25° C. and 160° C., more preferentially between 40° C. and 120° C. Although it is possible to implement the method according to the invention at a pressure greater than atmospheric pressure, it is most frequently preferred to operate at atmospheric pressure.

The copolymerisation reaction time in step (a) can vary from 30 min to one or more hours, or even one to several days, especially when no solvent is used. Tracking of the progress of the reaction by sampling and evaluating the product formed by the proton nuclear magnetic resonance (¹H NMR) and/or Fourier Transform Infrared (FTIR) spectroscopy technique enables those skilled in the art to readily determine the most suitable reaction time for the conditions used.

At the end of the copolymerisation reaction in step (a), the reaction medium can undergo various known separation or purification techniques such as: water treatment for example to remove the catalyst used, hydrolysing unreacted isocyanates groups; extraction with an organic solvent wherein the copolymer formed can be dissolved such as dichloromethane or chloroform; evaporating the reaction and/or extraction solvent(s); and vacuum drying.

In practice, the copolymer retrieved at the end of step a) is dissolved in a suitable solvent such as dichloromethane or chloroform, then precipitated by adding a non-solvent such as water, ethanol, 1-propanol, diisopropyl ether, 2-butanol, hexane or one of the mixtures thereof, cold (temperature below 20° C.), vacuum-dried or freeze-dried and stored, preferably in argon or nitrogen in a sealed container.

The copolymer obtained according the present invention has advantageously a mean molecular weight greater than 100000 g/mole, preferentially, a mean molecular weight ranging from 250000 g/mole to 400000 g/mole.

The porous structure of the polymer matrix according to the present invention, can be created with any technique known to those skilled in the art. By way of example, mention can be made of the NaCl salt particle leaching technique described in the patent documents US 2007/0015894 and WO2009/141732. Adapted to the copolymer according to the present invention, this technique consists of the following steps:
step 1): preparing a homogeneous solution of the copolymer in a suitable solvent wherein the copolymer is soluble;
step 2): introducing, under stirring, into the homogeneous solution obtained in step 1), NaCl table salt in the form of particles wherein the diameter is between 50 microns and 400 microns, preferably between 100 microns and 300 microns, the ratio, by weight, of copolymer to salt being from 5:95 to 30:70, preferably about 10:90;
step 3): pouring the mixture obtained in step 2) into a mould, allowing this mixture to solidify to obtain a moulded product;
step 4): retrieving the moulded product obtained in step 3), then immersing one or more times in water to remove the NaCl salt particles and the copolymer solvent;
step 5): drying, for example at a temperature greater than 60° C., the porous polymer matrix obtained in step 5) or freeze-drying same

The suitable solvent used in step 1) is preferably a water-soluble organic solvent, such as tetrahydrofuran (THF), N—N-dimethylformamide (DMF), dioxane, dimethylsulfoxide (DMSO), and dimethylacetamide (DMAc), which can be removed readily by subsequent washing with water. The copolymer content (expressed as a % weight/volume) in the solution of step 1) can vary from 20% to 50%.

Step 1) and step 2) can be conducted at a temperature between 20° C. and 150° C., preferably between 25° C. and 120° C.

In step 2), instead of NaCl salt, particles (diameter from 50 microns to 500 microns) of any pore-forming agent which is suitable for generating porosity within the copolymer with a view to obtaining the polymer matrix, and which is non-toxic and insoluble in the copolymer solvent and suitable for forming pores can be used. By way of example of such a pore-forming agent, mention can be made of mineral salts such as potassium chloride, calcium chloride, sodium citrate and sugars such as, but without being limited thereto, glucose, fructose, dextrose, maltose and sucrose, and mixtures thereof.

The mould containing the mixture obtained in step 2) can be cooled to a temperature less than 20° C., preferably to a temperature less than 0° C., for example to a temperature of −20° C.

The water used to wash the moulded product one or more times can be a deionised water of ultrapure quality.

The polymer matrix according to the present invention has therefore a porous structure wherein the pores have a size which is advantageously between 25 microns and 500 microns, preferentially between 50 microns and 300 microns and wherein the porosity rate ranges, preferably, from 40% to 95% by volume, in particular from 50% to 85%. Advantageously, the pores of the polymer matrix are interconnected. The porous polymer matrix structure according to the present invention promotes good fibrocartilaginous tissue growth.

The size and distribution of the pores of the polymer matrix can be analysed with a scanning electron microscope (SEM).

Preferably, the polymer matrix or the copolymer forming same has a mean molecular weight greater than 100000 g/mole, preferentially, a mean molecular weight ranging from 250000 g/mole to 400000 g/mole.

The porous biocomposite material can have a density from 0.1 g/cm³ to 0.5 g/cm³.

The mechanical properties and the degradation rate of the porous polymer matrix (or of the biocomposite material, or of the implant to be produced comprising same) can be adjusted by playing with one or more of the following parameters:
the molar lactide/glycolide ratio in prepolymer B (poly(lactide-co-glycolide diol) used. Preferably, this molar lactide/glycolide ratio is from 75:25 to 25:75, preferentially, it is 50:50.
the molar ratio between prepolymer A (poly(ε-caprolactone) diol) and prepolymer B (poly(lactide-co-glycolide diol) included in the composition of the porous polymer matrix. This molar ratio of prepolymer A to prepolymer B ranges advantageously from 10:90 to 90:10;
the molecular weight of prepolymer A (poly(ε-caprolactone) diol) and that of prepolymer B (poly(lactide-co-glycolide diol). Preferably, for prepolymer A and prepolymer B, the molecular weight ranges from 600 g/mol to 15000 g/mol or greater than 15000 g/mol, preferably from 1000 g/mol to 10000 g/mol; more preferentially, from 1500 g/mole and 6000 g/mole. The molecular weight of prepolymer A can be greater than or less than that of prepolymer B (poly(lactide-co-glycolide diol). The molecular weight denotes the mean molecular weight;
the porosity rate of the porous polymer matrix.

Advantageously, the porous biocomposite material according to the present invention has a tear resistance as per the standard DIN ISO 34-1 B (b) of a value greater than 10 N/mm, preferentially, of a value between 20 N/mm and 25 N/mm; and a compressive strength between 2 MPa and 3 MPa; and an elongation at break as per the standard DIN EN ISO 1798 of a value greater than 300%, preferentially of a value between 350% and 500%.

The porous polymer matrix (or the biocomposite material, or the implant comprising) also has the advantage of being progressively resorbed after implantation in a (human or animal) patient such that a substantial part of the matrix is resorbed over 6 to 18 months, preferably 6 to 12 months, after implantation. This enables the new fibrocartilage to acquire resistance to joint pressures.

According to a further embodiment, the mechanical properties of the biocomposite material according to the present invention can advantageously be customised and approach those of the human meniscus, which is not the case of the implants used to date in clinical surgery (polyurethane Actifit, and Collagen bovine Menaflex CMI Stryker) which have low resistance and low colonisation levels by fibrocartilaginous tissue cells, with a particular fragility for the bovine collagen CMI which tears like a piece of wet blotting paper in a liquid medium. These two implants have been withdrawn from the market.

After step 5), described above, the dried or freeze-dried porous polymer matrix is generally stored in any type of known packaging or container suitable for being hermetically sealed, prior to the use thereof for producing the composite collagen/porous polymer matrix material according to the present invention. This packaging or this container containing the porous polymer matrix can further undergo sterilisation, for example by ionisation, by heating and/or by chemical treatment. Preferably, the sterilisation is carried out by ionisation with gamma or beta radiations, more preferably with beta radiation. In particular when the sterilisation is carried out by ionisation, the quantity of radiation absorbed is from 0.5 kGy to 50 kGy, preferably from 1 kGy to 27 kGy.

According to a preferred embodiment of the present invention, the mould used in step 2) described above, comprises a mould cavity which is designed to have approximately the shape and the dimensions of a medical device or implant to be produced, and the mixture obtained in step 2) has been poured into this mould cavity. By way of example of implants to be produced, mention can be made of the knee meniscus implant and the temporomandibular joint (TMJ) meniscus implant.

In particular, the porous polymer matrix according to the present invention has a 3D shape intended to imitate the anatomical shape of the lateral or medial meniscus of a patient's knee, particularly of a human patient's knee.

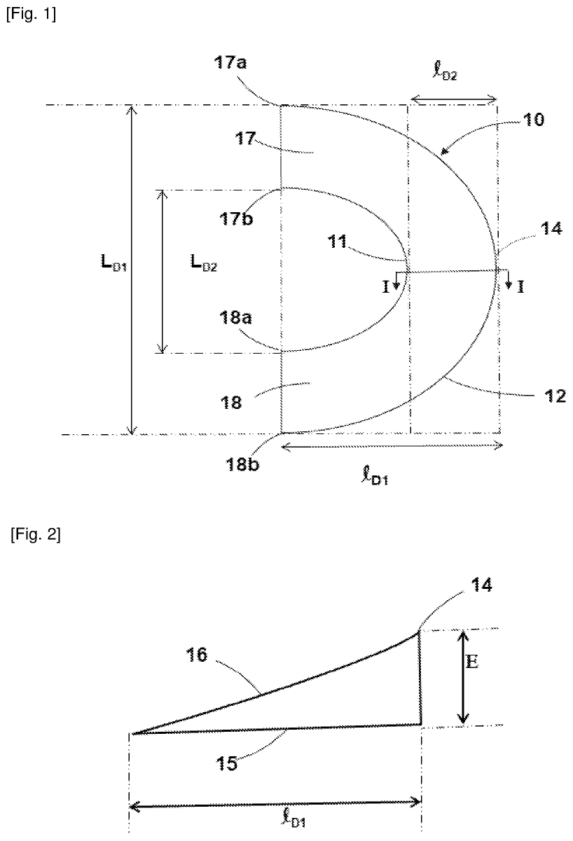

FIG. 1 illustrates a porous polymer matrix having a shape suitable for use as a knee meniscus replacement or repair implant. This porous polymer matrix has, generally, a C shape as illustrated in FIG. 1 and a wedge-shaped central cross-section, as seen in FIG. 2.

In FIG. 1, the C-shaped body 10 of the porous polymer matrix, comprises a concave perimeter edge 11 and a convex perimeter edge 12.

The convex perimeter edge 12 defines the ends 17, 18 of the C-shaped body 10 of the porous polymer matrix. The concave perimeter edge 11 and that of the convex perimeter 12 are opposite and spaced over the width of the body 10. The ends 17, 18 of the two branches of the body 10 can have a rounded or straight shape.

In FIG. 2, such a C-shaped body 10 has a flat bottom face 15, and an inclined, preferably slightly curved concave, top surface 16. The central part 14 of the convex perimeter edge 12 is much thicker than the concave perimeter edge 11. For example, this central part 14 can have a thickness E of about 13 mm, whereas the concave perimeter edge 11 is very thin with a thickness of less than 2 mm, preferably a feathered edge. The convex perimeter edge 12, at least in the central part 14 thereof, rises substantially perpendicularly to the bottom surface 15 of the body 10. The convex perimeter edge 12 retracts from the thick central part 15 thereof towards the ends 17 and 18 of the body 10 (see FIG. 2), such that the convex perimeter edge 12 retracts substantially towards the ends 17 and 18 in a feathered edge.

According to a preferred alternative embodiment of the present invention, the C-shaped body 10 of the porous polymer matrix or of the implant according to the present invention, has advantageously the following dimensions:
The distance between the respective ends 17b, 18b of the edges 17 and 18 of the body 10: L_{D1}=about 52 mm;
distance between the respective ends 17a, 18a of the edges 17 and 18 of the body 10: L_{D2}=about 28 mm;
The width of the central part 14: [symbol] =about 15 mm
thickness of the central part 15: E=about 13 mm;
length of the flat bottom face: [symbol] =about 28 mm.

In an alternative embodiment of the present invention, the body 10 of the porous polymer matrix has a single C shape readily applicable for damaged lateral and medial meniscus replacement or repair. The body 10 of the porous polymer matrix can for example be cut out to match exactly the dimensions of the lateral or medial meniscus to be repaired or replaced.

Preparation of the Porous Biocomposite Collagen/Polymer Matrix Material

The biocomposite material according to the present invention comprises the porous polymer matrix and collagen which coats the pore surfaces and the outer surfaces of the porous polymer matrix.

Advantageously, the ratio by weight of collagen to polymer matrix is from 20:80 to 40:60.

The application of collagen to the porous polymer matrix can be carried out with any technique known to those skilled in the art, for example:
by immersing the porous polymer matrix in collagen;
by chemical grafting (or cross-linking), for example with a grafting agent such as glutaraldehyde or carbodiimide CMC ((N-cyclohexyl-N0-2-morpholinoethyl-carbodiimide-methyl-p-toluolsulfonate), or others.
by thermal grafting (or cross-linking), for example at a temperature between 50° C. and 150° C., and for a duration between 1 h and 24 h
by 3D printing, in particular to coat the outer surfaces of the porous polymer material with several layers of collagen; and/or to spatially model the alignment and geometry of the collagen fibres; and/or to incorporate collagen in the porous polymer matrix.

It should be noted that the copolymer prepared by copolymerisation described above is terminated at one of the ends thereof with an amine function and at the other end with an alcohol function. The amine function results from the hydrolysis of one of the unreacted isocyanate groups of diisocyanate C to form a urethane structural unit. This amine function can be made used for the chemical grafting of collagen to the porous polymer matrix, via for example glutaraldehyde or carbodiimide CMC ((N-cyclohexyl-N0-2-morpholinoethyl-carbodiimide-methyl-p-toluolsulfonate).

The collagen fibres can be oriented so as to simulate the fibrous structure of native collagen. In particular, the collagen fibres can be incorporated in the porous polymer matrix while extending therefrom. This can be carried out readily, for example, with various known 3D printing techniques.

The collagen used within the scope of the present invention can be:
a human collagen, more specifically, a collagen sampled from the patient themselves, or
a non-human collagen, for example bovine collagen, or
a human recombinant collagen produced in genetically modified, prokaryotic or eukaryotic, cellular organisms,
a human recombinant collagen or a mixture of human recombinant collagens produced by genetically modified plants, these plants possibly being, particularly, rapeseed, tobacco, corn, pea, tomato, carrot, wheat, barley, potato, soya, sunflower, lettuce, rice, alfalfa, and beetroot.
a type I or II collagen or a combination thereof.

The collagen used within the scope of the present invention can be a combination of two or more of the collagens defined hereinafter.

The use of recombinant human or synthetic collagen in the biocomposite material according to the present invention is preferred in order to prevent an immune or allergic response to the collagen.

Preferably, the collagen used is a human recombinant collagen produced in genetically modified plants, preferentially, a human recombinant collagen expressed by tobacco plants.

The human recombinant collagen expressed by tobacco plants can be obtained with any known method in the prior art, in this regard, see, for example: the patent documents: WO1998027202A1 (Meristem Therapeutics S.A) and WO2006035442A2 (Collplant Ltd).

More preferentially, the collagen is a type I and type II human recombinant collagen which can be produced according to a specific method described hereinafter.

The biocomposite material or the implant according to the present invention can further comprise one or more types of live cells and/or one or more biologically active agents.

The population(s) of live cells to be incorporated into the biocomposite material or the implant according to the invention are preferably, of the type of those generally found in the type of tissue to be replaced or which can be differentiated into this type of cells. In particular as regards meniscal lesions or advanced, grade 3 and/or 4, arthrosis of the knee, these live cells can be chondroblasts, chondrocytes, stem cells, mesenchymal stem cells, adipose stem cells, embryonic stem cells, with the exception of embryonic stem cells of human origin. The population(s) of live cells of interest, in cell therapy, can be added to the biocomposite material or implant immediately prior to insertion into the implantation site of the (human or animal) patient's body, or can be inoculated and cultured on the biocomposite material (or implant) on the days or weeks prior to implantation. Alternatively, the population(s) of live cells of interest, in cell therapy, can be supplied to the implant (biocomposite material) after implantation. The techniques for preparing cell-filled implants are known in the art and are described for example in the patent document U.S. Pat. No. 6,103,255 (Rutgers State University of New Jersey, USA).

By way of non-limiting examples of biological active agents which can be added to the biocomposite materials (or the implant), mention can be made of anaesthetic agents, opacifying agents, anti-inflammatory agents, therapeutic agents, growth factors, blood platelets or combinations thereof.

Opacifying agents or contrast agents: are echogenic radio-opaque materials or materials sensitive to magnetic resonance imaging (MRI). They are useful for assisting with viewing the implant after implantation for example by ultrasonography and/or by MRI.

In practice, the biocomposite material or the implant produced according to the present invention is placed in a suitable moisture- and/or air-tight package or container suitable for protecting and storing same prior to the use thereof. Preferably, the biocompatible material or the implant is humidified then biologically sterilised and finally hydrated with a view to being packaged in the sterile state in packaging hermetically. By way of example of such packaging, mention can be made of aluminium foil blister type packs.

The porous polymer matrix impregnated with collagen according to the invention offers a favourable environment for cellular growth and for fibrocartilaginous tissue formation similar to the meniscus.

Thus, the porous biocomposite material according the present invention can be used for producing implants for the repair of a lateral or medial meniscus lesion of the knee; and/or treatment of a knee joint cartilage lesion; and/or prevention or treatment of knee arthrosis, particularly of grade 3 or 4 arthrosis of the knee; and/or regeneration of knee joint cartilage. Preferentially, the implant according to the present invention is embodied in a single C shape suitable for use for both the lateral meniscus and the medial meniscus.

The implant (or composite material) according to the invention can be inserted into the implantation site of a patient's body with surgical or arthroscopic techniques. In general, to implant the implant, the damaged tissue is removed at least in part, and the implant is inserted to replace or repair the tissue or the part of the tissue removed and is secured in place to the adjacent tissue at the implantation site, for example, by suture.

Collagen I and II Expression in the Tobacco Leaf

(1) Laboratories have created human recombinant collagen, most frequently type I (Collplant, Israel), or type II (Ruggiero); but few or none have created collagen of both types I and II at the same time as proposed by the invention. Type I is found in the skeleton and cartilage, and on the border of the meniscus, and type II in the rest of the meniscus, hence the benefit of having both type I and II human recombinant collagen.

Collagen is the most predominant protein of the extracellular matrix and connective tissue, and is involved in tissue growth, remodeling, repair, and overall physical support. There are more than 25 variations of collagen, but types I, II and III represent more than 80% of collagens in the body.

The properties of collagen are determined by the triple-helix structure thereof, formed by 3 alpha polypeptide chains wound along a common axis, and constructed by repeated Gly-XY triplets, where X and Y are frequently the amino acids proline and lysine.

(2) Two alpha 1 chains and one alpha 2 chain for type I collagen, heterotrimeric chain, of which the coding genes are Col 1A1 and Col 1A2; and three alpha 1 chains (II) for type II, homotrimeric chain, of which the coding gene is Col 2A1.

The collagen fibril is synthesised in the form of procollagen precursors assembled in the endoplasmic reticulum and containing propeptides with N and C-terminal spherical extensions.

During the translocation through the membrane of the endoplasmic reticulum, a post-translational process takes place: a hydroxylation dependent on prolyl-4-hydroxylase (P4H) and lysine hydroxylase 3 (LH3) of the proline and lysine residues in the repeated Gly-XY region, giving hydroxyproline and hydroxylysine. Thus procollagen undergoes several post-translational modifications before maturing to tropocollagen (collagen unit) which can be cross-linked into collagen fibrils. Thus, the co- and post-translation modifications, hydroxylation of proline and lysine, and the formation of intra- and inter-chain disulphide bonds, are required for triple-helix folding, assembly, and stability.

(3) Plants can produce proteins containing hydroxyproline; however, plant propyl hydroxylase can hydroxylate human collagen with difficulty. It is necessary to create plants to individually co-express type I (COL1A1 and COL1A2; hetero-trimer) and II (COL2A1; homo-trimer) collagen with human prolyl-4-hydroxylase (P4H) and human lysyl hydroxylase 3 (LH3), which can catalyse the three modification steps (lysyl residues to hydroxylysyl, galactosyl hydroxylysyl and glucosylgalactosyl hydroxylysyl) required for the formation of carbohydrates bound to hydroxylysine, to co-express recombinant proteins, monitored by gel electrophoresis and immunoblot, and mass spectrometry for the amino acid content.

(4) Plant transformation mediated by an Agrobacterium strain makes it possible to transfect much larger genetic sequences in plant cells, enabling the expression of procollagen1α1, complete procollagen1α2, procollagen 2 α1 as well as of the two prolyl hydroxylase and lysyl hydroxylase subunits in the same cell. Signal peptides were added to direct the accumulation of collagen in the subcellular compartments of the tobacco cell, such as the plant vacuole. The collagen produced by tobacco, thermally stable triple-helix structures, is capable of producing aligned fibrils, and has shown similar biofunctionality to collagen derived from human tissue, supporting the bonding and proliferation of endothelial progenitor type cells.

(5) According to a further aspect of the invention, the plant system is provided comprising a first genetically modified plant capable of accumulating an alpha 1 and alpha 2 chain of collagen and a second genetically modified plant capable of accumulating the alpha 1 chain of collagen. Combinations are possible, with a plant accumulating an alpha 1 chain, and a plant accumulating an alpha 2 chain.

(6) According to a further aspect of the invention, the method comprises the expression of the alpha 1 chain of collagen in the first plant; and the expression of the alpha 2 chain of collagen in a second plant; the first plant and the expression in a second plant of the alpha 1 chain of collagen and of the alpha 2 chain of collagen; the methods for producing firstly a fibrous collagen, by selecting a descendant expressing the alpha 1 chain of collagen and the alpha 2 chain of collagen are provided.

The method further comprises the expression of an exogenous P4H and of LH3 in each of the first and second plants. These steps are carried out by expression in plant organelles containing DNA.

(7) The present invention of embodiment of RH Collagen, envisages genetically modified tobacco plant cells co-expressing both human procollagen and a P4H and LH3, capable of correctly hydroxylating the alpha 1 and 2 chains of procollagen [i.e. hydroxylating the proline position (Y) of the Gly-XY triplets], and lysine.

RhCollagen has shown a superior biological function compared to any collagen derived from tissue, whether from animal or human tissue according to the data published in peer-reviewed scientific publications. RhCollagen can be manufactured in different forms and viscosities, including gels, pastes, sponges, sheets, membranes, fibres, and thin layers, which have all been tested in vitro and on animal models and proved to be superior to tissue-derived products.

Due to the homogeneity thereof, RhCollagen can produce fibres and membranes of high molecular order, which means that all the molecules are oriented in the same direction, which enables the formation of tissue repair products with distinctive physical properties, particularly a superior tensile strength due to the alignment of the collagen fibres, in respect of transparency, and the ability to attain high collagen concentrations at low viscosities.

Type I and II recombinant human collagen, RhCollagen I and II, is identical to the collagen produced by the human body.

The main advantages of products using RHCollagen, compared to those using collagen derived from animals or human cadaver tissue, comprise: speed in cell proliferation and tissue repair; a fully functional 3D matrix; perfect alpha collage helices; high cellular bonds.

RhCollagen has a superior biological function compared to collagen derived from animal or human tissue and has a number of useful physical characteristics, including thermal stability or resistance to decomposition at high temperatures, and a primary triple-helix, according to some publications. A perfect triple-helix enables superior bonding, which accelerates the proliferation of primary human cells, fibroblasts, and fibrocartilaginous tissue.

In all the cell types tested, cell proliferation was significantly superior in the scaffold made of rhCollagen than in commercially available scaffolds made of bovine collagen. The accelerated cell proliferation obtained with rhCollagen induces quicker healing, and better-quality tissue regeneration.

Pure rhCollagen does not induce an immunogenic response, whereas the impurities from the tissue-derived collagen source can result in immune system rejection. In vitro studies conducted within the scope of an academic collaboration have shown that rhCollagen incubated with activated THP1 macrophages produces significantly lower levels of inflammatory cytokines compared to bovine collagen. This demonstrates that animal-derived collagen can trigger a foreign body reaction not observed with rhCollagen, which delays healing and increases scars. Furthermore, there are no potential side-effects in tissue growth, as there is no growth residue. The factors originate from the extracted tissue. Furthermore, with tissue-derived collagen, it is possible that the animal or human from which the collagen was produced may have been infected with a virus, a prion, or another pathogenic agent. With rhCollagen, there is no risk of transmission of diseases and pathogens.

RhCollagen is synthesised by several human genes in tobacco plants producing pure molecules which are repeatable and identical to human type I and II collagen; it is more homogenous than collagen derived from animal or human tissue sources. The high level of homogeneity of RhCollagen enables the formulation of extremely high concentrations of soluble triple-helix type I and II collagen, up to 150-200 mg/ml, which is at least 10 to 100 times higher than the concentration obtained with tissue-derived collagen. The high concentration of homogeneous monomeric collagen is particularly important with solid collagen fibres are required for 3D scaffolds. The homogeneity of RhCollagen makes it possible to prepare consistent and reproducible products with a controlled degradation rate which can be optimised for the targeted indication.

Compared to tissue-derived collagen, RhCollagen membranes showed a superior thermal stability, a superior tensile strength due to the alignment of the collagen fibres, and higher levels of transparency. Type I Rh Collagen has been authorised for human use by the EMA (Europe) and the FDA (USA).

According to an embodiment, the meniscal implant is arched: the meniscal implant has a single both medial and lateral, C or crescent shape, the cross-section whereof is wedge-shaped; and the elasticity whereof makes it possible to modify same to adapt to the shape of the medial or lateral meniscus treated. It is concave at the top part thereof and planar in the bottom part thereof. FIG. 1 notes the antero-posterior dimensions, and FIG. 2 the medio-lateral dimensions and the thickness. It is thicker and wider than the implants of the prior art. The cross-section thereof is wedge-shaped and the surface thereof is planar on the tibial side and curved on the femoral side. The meniscal implant has incorporated suture systems, enabling more rapid suture on the meniscal wall or the peripheral capsule.

In an embodiment, the invention is a composite meniscal scaffold implant, indicated and used for grade 3 or 4 terminal knee arthrosis with at least 50% meniscal loss, and according to inclusion criteria (age, weight, physical fitness), to enable cartilage regeneration combined with cell therapy; and consisting of a mechanical structure made of high-molecular-weight polyurethane with Lysine Diisocyanate LDI, and PLGA copolymers, made of foam and porous; and of a biological structure stimulating the cellular and fibrocartilaginous tissue colonisation of the scaffold, including layers of collagens, Rhcollagens, non-animal, type I and II human recombinant collagen, expressed by the tobacco plant.

One of the embodiments of the invention relates to biocompatible meniscal implants manufactured from porous polyurethane foams, polymers, and recombinant plant collagen of the present method; they are degraded after implantation and the degradation products are biocompatible. Biopsies at 12 months were carried out, showing maturing tissue with fibrochondrocytic differentiation and organised collagen bundles. Clinical studies have been published, showing the incorporation of the Actifit polyurethane meniscal implant (Orteq) under MRI and second-look arthroscopy, and good clinical outcomes (Verdonk, Assor). However, partial degradations and polyurethane fragments; signs of toxicity, and spontaneous destruction resorption of the Menaflex bovine collagen scaffold, have been observed as well as cellular colonisation insufficiency, and underlining the need for a meniscal scaffold implant adapted to the knee joint environment.

In an embodiment, the mechanical and biological composite meniscal implant is manufactured by a 3D printer.

The present invention also relates to a method wherein the collagen is a type I and II human recombinant collagen.

Method for manufacturing type I and II human recombinant collagen, wherein two types of human collagens, type I and II, are generated, expressed by tobacco plants where corresponding genetic codes have been incorporated, the alpha 1 and 2 chains of the helices of type I and II collagens are expressed, and P4H and human exogenous LH3 are accumulated and expressed, for alpha chain hydroxylation; and collagen is generated from human procollagen which has accumulated in the vacuole of a plant; said method comprising contacting the human procollagen with propeptides, with a protease such as ficin during or after human procollagen extraction from a cell of said plant, digesting the propeptides, thus generating collagen.

Method, wherein:
a first genetically modified plant is provided, capable of accumulating an alpha 1 and alpha 2 chain of Col1A1 and Col1 A2 collagen;
a second genetically modified plant is provided, capable of accumulating a second alpha 1 chain of Coll2A1, further comprising a P4H and exogenous LH3;
the first genetically modified plant is crossed with the second genetically modified plant, to obtain a third genetically modified plant capable of expressing the two alpha 1 and alpha 2 chains of fibrous collagen.

Method, wherein the type I and II human recombinant collagen has sufficient temperature stability characteristics for the accumulation thereof in the third genetically modified plant.

Method, wherein the type I and II human recombinant collagen has sufficient temperature stability characteristics for the accumulation thereof in the third genetically modified plant.

Method, wherein the type I and II human recombinant collagen presented preferentially in fibril form is extracted from the third genetically modified plant and then purified.

Method, wherein the first and the second genetically modified plants are tobacco plants.

Method, wherein the type I and II human recombinant collagen is used in medicine and in regenerative surgery; in particular in cartilage regeneration; or incorporated in a porous polymer matrix by soaking, chemical cross-linking; thermal cross-linking or 3D printing.

Mechanical and Biocompatibility Tests
1. Determining of molecular weight of polyurethane and polymers. The molecular weight of the polymers is determined using Gel Permeation Chromatography (GPC) (Shimadzu T030845) with polystyrene standards and using 0.01 M LiBr in DMF
2. Tear resistance and flexibility of foam. Tests are performed on samples of about 10 mm in thickness. 2-0 sutures were positioned 3-5 mm from the edge of the sample, placed in the grips of the tensile tester (Instron 3342). The transversal head speed was 10 mm/min. The tear resistance is calculated as follows: the maximum force (N) is divided by the test sample thickness. The tear resistance is at least 3.0 N/mm, preferably from 20 to 25 N/mm.
3. The flexibility was calculated as follows: the displacement at break divided by the distance from the suture to the edge of the implant material.
4. Foam density: On samples of about 8-10 mm in thickness, the dimensions and the weight of the sample were determined and the density (g/cm3) was calculated based on the material mass (g) and volume (cm³).
5. Porous scaffold degradation. In vivo degradation is envisaged to take place over an average period of 4 years. According to the tests of the prior art and in particular the tests of the Orteq polyurethane patent, changes in molecular weight during in vitro degradation at 37° C. in a phosphate buffer demonstrated, after 1.5 years, that the molecular weight decreased to 50% of the original molecular weight thereof.
6. Implant cytotoxicity: This was shown to be non-toxic for the copolymer with di-urethane units derived from LDI (lysine diisocyanate). A segment of a polymer implant was extracted and the extract was placed in contact with cells. Cell lysis (cellular death), cellular growth inhibition and other effects on the cells caused by the extract were determined. The implant passed and there was no evidence of cell lysis.
7. Similarly, absence of allergic or irritation reaction after injecting the product via the intradermal route in rabbits; or of systemic toxicity after intraperitoneal injections in mice.
8. Genotoxicity on implant: bacterial reverse mutation. The test was carried out to evaluate the mutagenic potential of the Orteq patent polyurethane implant. The bacteria were exposed to the implant extracts; the mutation was determined after incubation. The implant according to the present invention demonstrated an absence of toxic effect.
9. Genotoxicity on implant: chromosome aberration on mammalian cells in vitro. The test was carried out to evaluate the potential clastogenic properties on human lymphocyte chromosomes. Human lymphocyte cultures were exposed to the implant extracted in 0.9% NaCl. A preliminary study was carried out without the metabolic activation system in order to determine the possible toxicity of five concentrations of the extract. The highest non-toxic concentration (40 μL of extract/mL of culture medium) was tested. After the contact period, the cultures were treated in order to prepare the chromosomes. The detection of aberrations was carried out by observing the chromosomes. The plant passed, no effect was observed.
10. Genotoxicity on implant: mouse bone marrow micronucleus. The test was carried out to evaluate the mutagenic potential after intraperitoneal injections of the implant extracts in mice. The test and the negative control groups received an intraperitoneal injection for two days, whereas the positive control mice received a single intraperitoneal injection of cyclophosphamide the second day. Mice were observed immediately after the injection for the general state of health and for any adverse effect. On day 3, all the mice were weighed and terminated. The femurs were excised, the bone marrow was extracted and double smear preparations were produced on each. Mammalian cells were exposed to the implant extracted in 0.9% NaCl and in 96% ethanol. The mutation was determined after incubation. The implant passed, no mutagenic/toxic effects were observed.
11. Combined subchronic toxicity study and local tolerance study on an implant material and an accelerated implant (polyurethane segments). Accelerated implant degradation products were manufactured as follows. The powder implant material was subjected to 9M HCl for 3 days. The remaining material (hard segments) was isolated via several washing steps, centrifuged, and dried. An additional purification was performed by washing with pyrogen-free water and finally washing with 96% ethanol (pharmaceutical grade). After drying in a vacuum oven, the hard segments were milled with a motor and a pestle. The Malditov- and 1H-NMR analysis demonstrated that the degradation of the soft segment was effective and that the hard segments essentially remained. The SEM analysis was too large and not representative of the actual size of the hard segments (the small clustered particles following the washing and drying process). Consequently, a sonication procedure was carried out in a 0.9% saline solution, which gave a milky dispersion wherein, at rest, no sediment was observed. The SEM analysis revealed that 98% of the particles in the milky dispersion were representative of the size of the hard segments which would be expected after in vivo degradation of the hard segments (70 to 130 nm). The milky dispersion (0.4 ml) was injected into the dorsal subcutaneous space of rats and the site was marked by ink tattooing to identify the injection site at necropsy. Furthermore, disks of implant material weighing 90±2 mg of a thickness of 2.5±1.1 mm were sterilised and implanted in one side of the back of 10 male rats and 10 female rats (on the other side of the back 2 mL of 0.9% NaCl was injected by way of control). A control group received a disk of high-density polyethylene. The rats were observed immediately after implantation and every day afterwards to detect mortality or morbidity and any normal clinical signs. Body weight and dietary intake were recorded each week. At the end of the implantation interval (13 weeks), blood samples were drawn for haematology and clinical chemistry and the rats underwent a submacroscopic necropsy and a microscopic examination of selected organs and implanted sites. No mortality or clinical sign capable of being linked with a toxic effect of the implants was observed. The degraded implant material (hard segments) was absorbed by the macrophages.
12. Combined chronic toxicity and local tolerance study on an implant material and an accelerated implant (polyurethane segments), 26 weeks. A group of rats was implanted with the implant according to the present invention. A group was injected with the accelerated degraded implant (agglomerates of polyurethane segments of sizes 70-130 nm) as described above. A control group of 10 male rats and 10 female rats received a disk of high-density polyethylene. The rats were observed immediately after implantation, then daily to detect mortality or morbidity and any abnormal clinical sign. Body weight and dietary intake were recorded once a week. At the end of the implantation interval (26 weeks), blood samples were drawn for haematology and clinical chemistry and the rats underwent a macroscopic necropsy and a microscopic examination of the selected organs and the implanted sites. The implant passed, no clinical signs of toxic effects were observed. The degraded implant material (hard segments) underwent macrophage phagocytosis.
13. Wear debris analysis. The stress to which the knee is subjected is very high and implant particles can become detached from the implant. A wear debris test for the polyurethane polymer implants was performed in the rabbit knee model to demonstrate the safety of the particle debris. This test was carried out to evaluate the local tolerance of the wear debris resulting from implantation, four weeks after an intra-articular injection into the rabbit's knee. Four weeks after the injection, each knee was dissected, opened, and examined and a macroscopic examination of each compartment of the knee was carried out. No signs of pain or swelling and no accumulation of synovial fluid were observed.
14. Implantation in sheep or goats: the tests on live animals will be carried out to evaluate the present invention of composite polyurethane-polymer and recombinant plant collagen scaffold. According to the following procedure, the results whereof will be published.
   Loss of meniscal mass, after meniscectomy for severe lesions, or due to the arthrosic process with grade 3 or 4 (medial and/or lateral) femoro-tibial cartilage loss, is responsible for deterioration of knee arthrosis, (23): Scheller et al., Arthroscopy 17: 946-52 (2001). The porous implants according to the present invention will be studied to evaluate the long-term performances thereof after implantation in a partial ovine meniscectomy model on goats. Ten mature goats underwent a unilateral total meniscectomy. In 5 animals, the total meniscectomy was replaced by a scaffold. The primary outcome measured was the histological classification of the cartilaginous lesions on the tibial plateau and the femoral condyle. Secondary outcomes: (i) general appearance of the knee, (ii) scaffold friction coefficient over time, (iii) evidence of tissue penetration in the scaffold, and (iv) load transfer characteristics over time. Three months post-implantation, 5 implanted knees on a scaffold and 5 partially meniscectomised knees are analysed. Three months later, the cellular colonisation of the scaffold will be analysed. The cartilage damage is analysed; to demonstrate that cartilage protection is provided by the scaffold compared to partial meniscectomy without scaffold implantation.
15. A draft human clinical trial application will be filed with the health authorities

1-25. (canceled)

26. A porous biocomposite material comprising: a polymer matrix having pores defined by several surfaces, and collagen which at least partially covers the surface of the pores and the outer surfaces of the polymer matrix, wherein:
the polymer matrix comprises a copolymer which is a reaction product of a mixture comprising:
a prepolymer (A) which is a poly(ε-caprolactone) diol;
a prepolymer (B) which is a poly(lactide-co-glycolide) terminated by a hydroxyl group at both ends of the molecules thereof and which has a molar ratio of lactide to glycolide ranging from 75:25 to 50:50; and
a diisocyanate (C) which is a C1 to C4 alkyl ester of lysine diisocyanate;
and, optionally a catalyst;
the molar ratio between prepolymer (A) and prepolymer (B) in the mixture being from 10:90 to 90:10; and
the molar quantity of diisocyanate (C) in the mixture being approximately once the total quantity of prepolymer (A) and prepolymer (B); and
the ratio, by weight, of collagen to polymer matrix is from 20:80 to 40:60.

27. The porous biocompatible material according to claim 26, wherein the material is bioresorbable, biocompatible and biodegradable.

28. The porous biocomposite material according to claim 26, wherein prepolymer (A) and prepolymer (B) are biocompatible and biodegradable and each have a molecular weight ranging from 600 g/mol to 15000 g/mol or greater than 15000 g/mol.

29. The porous biocomposite material according to claim 26, wherein prepolymer (B) has a molar ratio of lactide to glycolide of 50:50.

30. The porous biocomposite material according to claim 26, wherein the pore size of the polymer matrix is between 25 microns and 500 microns.

31. The porous biocomposite material according to claim 26, wherein the polymer matrix has a porosity from 40% to 95% by volume.

32. The porous biocomposite material according to claim 26, wherein the polymer matrix has a mean molecular weight greater than 100000 g/mole, and a mean molecular weight ranging from 250000 g/mole to 400000 g/mole.

33. The porous biocomposite material according to claim 26, wherein the collagen is a human recombinant collagen produced in genetically modified plants.

34. The porous biocomposite material according to claim 26, wherein the collagen is a type I or II collagen or a combination thereof.

35. The porous biocomposite material according to claim 26, wherein it further comprises one or more types of live cells.

36. The porous biocomposite material according to claim 35, wherein the live cells are selected in the group consisting of chondroblasts, chondrocytes, stem cells, mesenchymal stem cells, adipose stem cells, where said stem cells are not human embryonic stem cells.

37. The porous biocomposite material according to claim 26, wherein the material further comprises at least one bioactive agent.

38. The porous biocomposite material according to claim 26, wherein the porous polymer matrix thereof is presented in the form of a moulded body which has the shape of a lateral or medial meniscus of the knee.

39. The porous biocomposite material according to claim 26, in a form suitable for use as an implant for the repair of a lateral or medial meniscus lesion of the knee, and/or prevention or treatment of a joint cartilage lesion of the knee, and/or prevention or treatment of knee arthrosis, particularly of grade 3 or 4 arthrosis of the knee.

40. An implant for the repair of a knee meniscus lesion; and/or treatment of a knee joint cartilage lesion; and/or prevention or treatment of knee arthrosis, particularly of grade 3 or 4 arthrosis of the knee; and/or regeneration of knee joint cartilage, wherein the implant comprises a biocomposite material according to claim 26.

41. The implant according to claim 40, wherein the implant is formed in a single C shape suitable for use for both the lateral meniscus and the medial meniscus.

42. The implant according to claim 40, wherein the implant is progressively resorbed after implantation in a patient in 6 months to 18 months after implantation.

43. A method for manufacturing a biocomposite material according to claim 26, comprising at least the steps of:
providing a porous polymer matrix according to claim 26,
applying collagen to the polymer matrix to obtain the biocomposite material.

44. The method according to claim 43, wherein the collagen is applied to the porous polymer matrix by soaking, chemical cross-linking, or thermal cross-linking.

45. The method according to claim 43, wherein the collagen is a type I or II human recombinant collagen or a combination thereof.
